(19) United States
(12) Patent Application Publication
(10) Pub. No.: US 2009/0030350 A1
(43) Pub. Date: Jan. 29, 2009

(54) GAIT ANALYSIS

(75) Inventors: Guang-Zhong Yang, Surrey (GB); Benny Lo, London (GB)

Correspondence Address: BROOKS KUSHMAN P.C., 1000 TOWN CENTER, TWENTY-SECOND FLOOR, SOUTHFIELD, MI, 48075, US

(73) Assignee: IMPERIAL INNOVATIONS LIMITED, London (GB)

(21) Appl. No.: 12/278,216

(22) PCT Filed: Feb. 2, 2007
(86) PCT No.: PCT/GB07/00358

§ 371 Date: Aug. 4, 2008

(30) Foreign Application Priority Data
Feb. 2, 2006 (GB) 0602127.3

(51) Int. Cl. A61B 5/103

(52) U.S. Cl. 600/595

(57) ABSTRACT

A method and system for analysing gait patterns of a subject by measuring head acceleration in vertical direction. The system comprises an accelerometer mounted on the head of the subject. The analysis includes calculating a signature from the acceleration data, using a Fourier transform, including energy of the first harmonics and comparing the signature with the baseline signature. Baseline signature is a representative of previously stored signatures. The comparison is done in order to monitor changes in the gait signatures over time. The entropy of the signatures may be used to perform the comparison. A self organised map is used to classify the measured gait signals.

The present invention relates to a method and system of analysing gait.

In analysing gait it is often desirable to monitor gait patterns pervasively, that is in a subject's natural environments in contrast to relying on a subject walking on a treadmill in front of a video camera. Known pervasive gait analysis systems typically place sensors on the ankle, knee or waist of the subjects, aiming to capture the gait pattern from leg movements. However, due to variation in sensor placement, these systems often fail to provide accurate measurements or require extensive calibration for detecting predictable gait patterns, for example abnormal gait patterns following an injury.

The inventors have made the surprising discovery that efficient gait analysis can be performed using an accelerometer placed on a subject's head, for example using an ear piece. Such an ear piece can be worn pervasively and can provide accurate measurements of the gait of the subject for gait analysis, for example in the study of recovery after injury or in sports investigations.

The invention is set out in independent claims 1 and 10. Further, optional features of embodiments of the invention are set out in the remaining claims.

The analysis may include detecting certain types of gait patterns by comparing a signature derived from the sensed head acceleration to one or more base line signatures. It may also include monitoring the historical development of a gait pattern of a subject by storing signatures derived from the acceleration signals and compare future signatures against one or more of the stored signatures (the stored signatures thus acting as the baseline).

Preferably, the acceleration sensor senses head acceleration in a substantially vertical direction when the subject is in an upright position. This is believed to measure the shockwaves travelling through the spine to the head as the subject's feet impact on the ground during walking or running.

The acceleration sensor may be mounted on the head in a number of ways, for example in an ear piece to be placed inside the outer ear, a hearing-aid-type clip to be worn around and behind the ear, or an ear clip or ear ring to be worn on the ear lobe. Alternatively, the acceleration sensor may be secured to another form of head gear for example, a headband or a hat, a hearing aid or spectacles, and may in some applications be surgically implanted.

The signature can be derived from the acceleration signal using a number of techniques, for example a Fourier transform or wavelet analysis. The signature may be analysed in a number of ways including calculating its entropy, using it as an input to a self-organised map (SOM) or a spatio-temporal self-organised map (STSOM), as described in more detail below.

An exemplary embodiment of the invention is now described with reference to the attached drawings, in which:

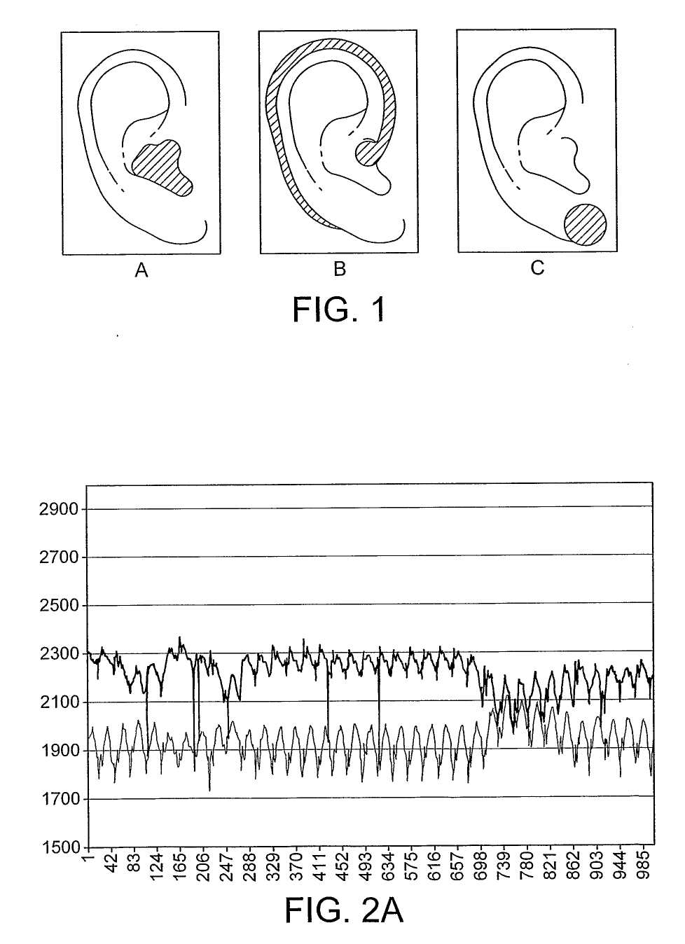

FIGS. 1A to C schematically show a number of different ways of attaching the acceleration sensor to a subject's head;

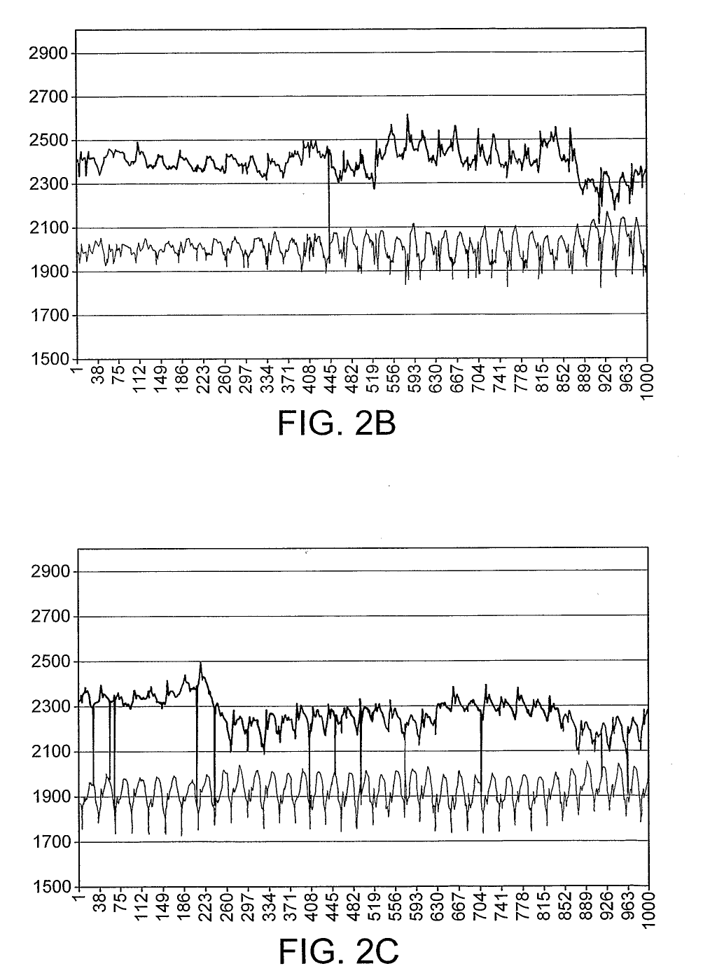

FIGS. 2A to C show acceleration data obtained using an embodiment of the invention for a subject before and after injury and when recovered; and

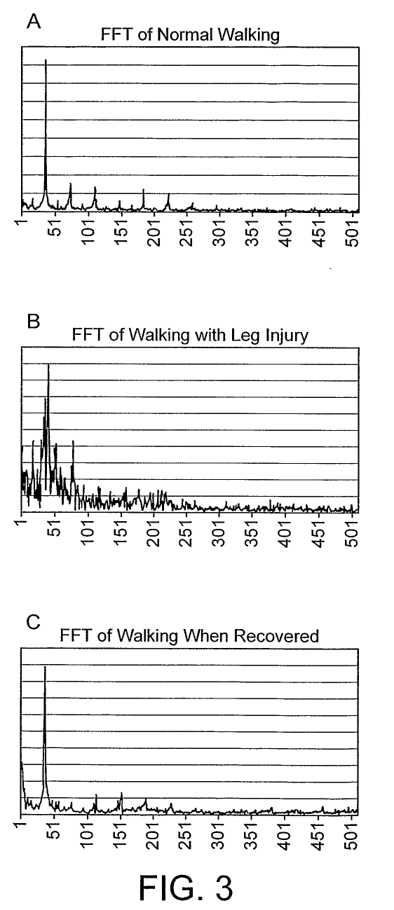

FIGS. 3A to C show plots of the corresponding Fourier transform.

FIGS. 1A to C illustrate three different housings for an acceleration sensor to measure head acceleration (A: earplug; B: behind-the-ear clip; C: ear clip or ring). Inside the housing an acceleration sensor is provided, coupled to a means for transmitting the acceleration signal to a processing unit where it is analysed. Additionally, the housing may also house means for processing the acceleration signal, as described in more detail below. The result of this processing is then either transmitted to a processing unit for further processing or may be stored on a digital storage means such as a flash memory inside the housing. While FIGS. 1A-C show different ways of mounting an acceleration sensor to a subjects' ear, alternative means of mounting the sensor to the head are also envisaged, for example mounting on a headband or hat or integrated within a pair of spectacles or head phones.

The acceleration sensor may measure acceleration along one or more axes, for example one axis aligned with the horizontal and one axis aligned with the vertical when the subject is standing upright. Of course, a three axis accelerometer could be used, as well.

It is understood that the housing may also house further motion sensors such as a gyroscope or a ball or lever switch sensor. Furthermore, gait analysis using any type of motion sensor for detecting head motion is also envisaged.

FIGS. 2A to C show the output for each of two axes for such an acceleration sensor worn as described, with the dark trace showing the horizontal component and the lighter trace showing the vertical component. The y-axis of the graphs in FIGS. 2A to C shows the measured acceleration in arbitrary units and the x-axis denotes consecutive samples at a sampling rate of 5O Hz. As is clear from the cyclical nature of the traces, each of the figures shows several footstep cycles.

The present embodiment uses the vertical component of head acceleration (lighter traces in FIGS. 2A to C) to analyse gait. It is believed that this acceleration signal is representative of the shock wave travelling up the spine as the foot impacts the ground during walking or running. This shockwave has been found to be rich in information on the gait pattern of a subject.

For example, in a healthy subject, gait patterns tend to be highly repetitive as can be seen in FIG. 2A showing the acceleration traces for a healthy subject. By contrast, in FIG. 2B, which shows acceleration traces of a subject following an ankle injury, it can be seen that following the injury the acceleration traces become much more variable, in particular for the vertical acceleration (lighter trace). It is believed that this is associated with protective behaviour while the subject walks on the injured leg, for example placing the foot down toes first rather than heel first followed by rolling of the foot as in normal walking.

FIG. 2C shows acceleration traces from the same subject following recovery and it is clear that the repetitive nature of, in particular, the vertical acceleration trace that regularity has been restored.

Based on the above finding, the detection of a gait pattern representative of an injury (or, generally, the detection of a gait pattern different from a baseline gait pattern) may be achieved by suitable analysis of the above described acceleration signals. In one embodiment, the vertical acceleration signal is analysed using a Fourier transform for example, calculated using the Fast Fourier Transform (FFT) algorithm with a sliding window of 1024 samples. The abnormal gait pattern can then be detected from the frequency content.

FIGS. 3A to C show the FFT for the respective acceleration measurements of FIGS. 2A to C. The y-axis is in arbitrary units and the x-axis is in units of (25/512) Hz, i.e. approximately 0.05 Hz. While the absolute value of the energy of the FFT (plotted along the y-axis) will depend on factors such as the exact orientation of the acceleration sensor with respect to the shockwave travelling through the spine and its placement on the head, as well as the overall pace of the gait, the plots clearly contain information on the type of gait pattern in the relative magnitudes of the energy of the FFT at different frequencies. It is clear that the relative magnitudes of the FFT peaks have changed.

As can be seen from FIG. 3A, the FFT of the acceleration signal of a healthy subject shows a plurality of, decaying harmonics. By contrast, the leg injury data (FIG. 3B) shows a much broader frequency content in which the spectrum lacks the well defined peaks of FIG. 3A and the non-uniform harmonics indicate abnormal gait. FIG. 3C shows the FFT of acceleration data for the same subject following recovery, and it can be seen that, to a large extent, the pre-injury pattern has been restored.

Summarising, a signature indicative of the gait pattern can be derived from the acceleration data and used to classify the gait pattern for example as normal or injured as above as demonstrated by the above data. In the above example, the signature is a Fourier transform. It is understood that other ways of calculating a signature are equally envisaged. For example, a signature can be calculated using wavelet analysis, for example by passing the data through a wavelet transform (e.g. first order Debauchies) and then using the transformed data as an input to a classifier, e.g. a SOM. For example, only the first high frequency component of the wavelet transfer could be used as an input to the classifier.

Once a signature is derived as described above, it can be analysed automatically in order to detect changes in the gait pattern. On the one hand, it may be desirable to detect whether the gait pattern is close to a desired gait pattern. This can be useful for example in training athletes. To this end, a signature obtained from acceleration data of a subject, for example an athlete, is obtained and compared to a baseline signature obtained from baseline data representing desired behaviour. The resulting information may then be used to, help an athlete in his training, for example helping a long distance runner to adjust his leg movements.

On the other hand, it may be desirable to use the above analysis to detect changes over time within a subject. For example, this can be useful in pervasive health monitoring where the gait pattern of a patient can be monitored such that a doctor or healthcare professional can be notified when a change in the gait pattern indicative of an injury is detected.

For example, one measure that can be used to detect changes in the signature is to calculate the entropy of the signature. In the example of the FFT described with reference to FIGS. 3A to C, it is clear that the entropy value for the injury data would be much larger than the entropy value for the normal data.

One way to compare and classify signatures is to use them as an input for a self organized map (SOM). For example, the energies of the FFT at the first four harmonics can be used as an input vector to an SOM. A person skilled in the art will be aware of the use of SOM for the analysis and clarification of data and the implementation of an SOM to analyse the signature as described above is well within the reach of normal skill of the person skilled in the art. Briefly, the SOM is presented with input vectors derived from the signatures described above during a training period for a sufficiently long time to allow the SOM to settle. Subsequently, activations of the output units of the SOM can then be used to classify the data. For example, it has been found that in a trained SOM data from the subject of FIGS. 2 and 3 may activate a first subset of units before injury and a second subset of units after injury.

In the embodiment described above, a signature is calculated using a sliding window FFT. As such, the resulting signature will be time varying such that more than one unit of an SOM will be activated over time. If it is desired to analyse the time varying nature of the input vector derived from the signature, an alternative analysis technique described in co-pending patent application WO2006/097734, herewith incorporated herein by reference, may be used. The application describes an arrangement, referred to as Spatio-Temporal SOM (STSOM) below, of SOMs in which, depending on the measure of the temporal variation of the output of a first layer SOM, a second layer SOM is fed with a transformed input vector which measures the temporary variation of the features in the original input vector. As in a conventional SOM, the output of the second, temporal layer SOM can then be used to classify the data based on its temporal structure.

Briefly, classifying a data record using an STSOM involves:
(a) defining a selection variable indicative of the temporal variation of sensor signals within a time window;
(b) defining a selection criterion for the selection variable;
(c) comparing a value of the selection variable to the selection criterion to select an input representation for a self organising map and deriving an input from the data samples within the time window in accordance with the selected input representation; and
(d) applying the input to a self organising map corresponding to the selected input representation and classifying the data record based on a winning output unit of the self organising map.

For example, the selection variable may be calculated based on the temporal variability of the output units of a SOM.

Training an STSOM may involve:
(a) computing a derived representation representative of a temporal variation of the features of a dynamic data record within a time window;
(b) using the derived representation as an input for a second self-organised map; and
(c) updating the parameters of the self-organised map according to a training algorithm.

The training may involve the preliminary step of partitioning the training data into static and dynamic records based on a measure of temporal variation. Further details of training an STSOM and using it for classification can be found in the above-mentioned published patent application.

It is understood that the sensor signals of the above described embodiment may also be used for human posture analysis and/or activity recognition. Furthermore, the system described above could be an integral part of a body sensor network of sensing devices where multiple sensing devices distributed across the body are linked by wireless communication links.

1. A method of analysing gait including measuring a signal representative of acceleration of the head of a subject whose gait is to be analysed, and applying a transform to the measured signal to compute a gait signature representative of the gait of the subject.

2. A method as claimed in claim 1 which further includes comparing the gait signature to a baseline signature to detect differences therebetween.

3. A method as claimed in claim 2 in which one or more signatures are stored over time and the baseline signature is representative of one or more stored signatures in order to monitor changes in the gait signature over time.

4. A method as claimed in claim 1 in which the measured signature is representative of an acceleration in a substantially vertical direction when the subject is in an upright position.

5. A method as claimed in claim 1 in which the transform is a Fourier transform.

6. A method as claimed in claim 5 in which the signature includes the values of the energy of the first n harmonics.

7. A method as claimed in claim 1 in which the transform is a wavelet analysis.

8. A method as claimed in claim 1 in which the signature is used as an input to a self organised map or a spatio-temporal self-organised map.

9. A method as claimed in claim 1 including calculating the entropy of the signature, and using the calculated entropy to compare signatures.

10. A gait analysis system including an acceleration sensor mounted in a sensor housing which is adapted to be secured to the head of a human: and an analyser operatively coupled to a sensor and operable to receive an output representative of head acceleration therefrom, and to apply a transform thereto for computing a gait signature representative of a gait pattern.

11. A system as claimed in claim 10 which further includes a comparator operable to compare the signature to a baseline signature in order to detect the differences therebetween.

12. A system as claimed in claim 11 which further includes a memory for storing one or more signatures of which the baseline is representative of one or more of the stored signatures such that the comparator can be used to monitor changes in the signature over time.

13. A system as claimed in claim 10 in which the housing is adapted to be mounted such that the output is representative of head acceleration in a substantially vertical direction when the subject is in an upright position.

14. A system as claimed in claim 10 which is included within the housing.

15. A system as claimed in claim 10 in which the housing includes an ear plug, a behind-the-ear clip, an ear ring, an ear clip, a hearing aid or a pair of spectacles.

16. A system as claimed in claim 10 in which the housing is secured to a headband, a hat or other head wear.

17. A system as claimed in claim 10, in which the transform is a Fourier transform.

18. A system as claimed in claim 17 in which the signature includes the values of the energy of the first n harmonics.

19. A system as claimed in claim 10 in which the transform is a wavelet analysis.

20. A system as claimed in claim 10 further including a further analyser including a self organised map or a spatio-temporal self organised map which is operable to receive the signature as an input.
